(19) United States
(12) Patent Application Publication
(10) Pub. No.: US 2004/0073189 A1
(43) Pub. Date: Apr. 15, 2004

(54) Vial access transfer set

(76) Inventors: Phil Wyatt, Glendale, CA (US); James D. Mullin, Glendale, CA (US); Gary Schaeffer, Glendale, CA (US); Freddy Zinger, Ra"Anana (IL)

Correspondence Address: James E. Brunton, Suite 860, 700 North Brand Blvd., P. O. Box 29000, Glendale, CA, 91203, US

(21) Appl. No.: 10/269,009

(22) Filed: Oct. 9, 2002

(51) Int. Cl. A61B 19/00; A61M 5/32

(52) U.S. Cl. 604/411; 604/403; 604/905

(57) ABSTRACT

A vial access transfer set for use with a bottle containing a medicinal fluid that provides a mechanism for securely attaching an administration set to the bottle and also provides a universal spike port to which any primary IV line can be readily attached. The vial access transfer set includes a novel, vented vial access adapter to which a length of tubing can be connected, the adapter comprising a top wall, a cannula connected to the top wall and a uniquely configured, resiliently deformable slitted skirt connected to the top wall and extending therefrom. With this construction, during the interconnection of the vial access transfer set with the bottle, the rubber septum of the bottle can readily be pierced by the cannula and the resiliently deformable skirt positively snapped into place over the neck portion of the drug vial or bottle to maintain the adapter in secure connection with the bottle. The apparatus is easy to use and positively prevents the accidental separation of the length of tubing carrying the medicinal fluid from the bottle.

### Background of the Invention

1. Field of the Invention

The present invention relates generally to intravenous (IV) liquid medicament infusion apparatus. More particularly the invention concerns a novel vial access transfer set for use with a vial containing a medicinal fluid.

2. Discussion of the Prior Art

A widely used method of medical therapy is the intravenous (IV) infusion of liquid medicaments and nutrients into the bloodstream of a patient. The apparatus that is used in the practice of this method typically comprises an IV container, such as an IV bag or bottle, which contains the liquid to be infused into the patient.

The IV bottle or vial typically comprises a container portion; an upper, generally cylindrically shaped portion within which a pierceable, septum like member is sealably mounted and an intermediate neck portion. During the infusion step, an IV spike is used to penetrate the septum to establish a pathway through which the fluid can flow to the patient. When the IV container is in the form of a flexible bag, the intravenous spike is pushed into a bag outlet port to establish a pathway through which the liquid can flow out of the bag. In either case, the spike is generally connected to or formed integrally with the inlet port of a small, elongated, transparent hollow container typically referred to as a “drip chamber”. With the construction thus described, a fluid communication pathway is formed between the medicament container and the inlet port of the drip chamber via a fluid pathway within the spike.

To enable fluid flow to the patient, an IV line is connected to the outlet port of the drip chamber. A flow control clamp such as a roller clamp, a slide clamp or other suitable flow-regulating device is typically engaged with the IV line to regulate fluid flow through the IV line. To complete the fluid flow path from the IV container to the patient, a needle is connected to the IV line to pierce the patient's tissue. The combination of the drip chamber, the outlet tube and the clamp is generally referred to as an “IV set”.

During the infusion of the medicament or nutrient to the patient, the bag or bottle is elevated above the patient to establish a positive pressure to force the fluid that is within the bag or bottle through the drip chamber into the patient. Following the interconnection of the IV set to the patient the medical technician typically establishes a predetermined flow rate of medicament into the patient by adjusting the roller or slide clamp on the IV line.

Modem-day therapy involves the infusion, in accordance with very strict protocols, of a variety of very expensive medicaments. For example, certain drugs can cost over $1000.00 per 100 cubic centimeters (cc). If the infusion of the medicament to the patient is accidentally interrupted, the result can be both expensive and in some cases catastrophic. At the present time, there is no reliable mechanical means for attaching the intravenous spike to the flex bag or to the rubber septum of the bottle and the spike is generally held in place only by the friction fit of the barrel portion of the spike into the flex bag or the rubber septum. Accordingly, in the typical hospital environment there is a finite possibility that the intravenous spike can become accidentally separated from the medicament container as a result of accidental entanglement with the IV set. This is particularly true if the IV spike is left in the rubber septum of the bottle for a long period of time. In such a situation, the septum tends to take a “set” permitting the medicament into a leak around the spike causing a potential loss of the medicament and at the same time making it easier for the spike to become accidentally separated from the septum.

Another drawback of the prior art methods for IV drug delivery resides in the fact that on many occasions the IV spike of the infusion set is of a length such that it penetrates into the drug bottle a distance that does not allow all of the drug to flow from the bottle during the infusion step. This often causes the nurse to manipulate the IV spike by moving it around in an attempt to drain the remaining drug from the bottle. This undesirable practice is generally ineffective and results in the loss of nursing time and possibly in the loss of a significant amount of the very expensive drug contained within the bottle.

It is the problems identified in the preceding paragraph that the apparatus of the present invention seeks to overcome. More particularly, the primary purpose of the vial access transfer set of the present invention is to provide a reliable means for securely attaching a conventional administration sent to a conventional drug vial or bottle. As will be a better understood from the description that follows, the vial access transfer set of the present invention uniquely functions to positively prevent the accidental separation of the infusion set from the bottle by providing a novel vial access adapter that ensures a secure interconnection of the IV set with the bottle. Additionally, the vial access transfer set of the invention provides a universal spike port to which any primary IV line can be simply and easily interconnected.

With respect to the prior art devices of which applicants are aware, U.S. Pat. No. 4,253, 501 issued to Ogle discloses a transfer device for the withdrawal of aliquots from a container of bulk solution. The transfer device is provided with the body having a tapered chamber, a first passage venting the smaller end of the chamber to the surrounding air, a second passage communicating at one end with the larger end of the chamber, and a third passage having one end for withdrawal of the fluid therefrom.

U.S. Pat. No. 4,588,503 issued to Weiss et al. discloses a vented syringe adapter assembly, which includes a housing having a sidewall, bottom wall, and top wall, which walls combine to define a chamber within the housing. A duel lumen tubular conduit extends through the top wall chamber and bottom wall with one end extending from the top wall and defining a syringe connector port and the opposite end extending from the bottom wall and defining a piercing tip. U.S. Pat. No. 6,238,372 B1 issued to one of the present inventors concerns a fluid control device for use with a syringe and at least one medicinal vessel. The fluid control device includes a first port, a second port for receiving the syringe, a third port, including in adapter having a fluid conduit member extending into the interior of the medicinal vessel when attached thereto and a flow control member selectively movable between first and second flow control positions.

The prior art patents identified in the preceding paragraphs, while pertinent to a general understanding of the present invention, fail to disclose or remotely suggest the novel construction of the vial access transfer set of the present invention as described in greater detail hereinafter.

### SUMMARY OF THE INVENTION

It is an object of the present invention to provide a vial access transfer set for use with a bottle containing a medicinal fluid that provides means for securely attaching an administration set to the bottle and also provides a universal spike port to which any primary IV line can be readily attached.

Another object of the invention is to provide a vial access transfer set of the aforementioned character that includes a novel, vented vial access adapter to which a length of tubing can be connected, the adapter comprising a top wall, a cannula connected to the top wall and a uniquely configured, resiliently deformable slitted skirt connected to the top wall and extending there from. With this novel construction, during the interconnection of the vial access transfer set with the bottle, the rubber septum of the bottle can readily be pierced by the cannula and, at the same time, the resiliently deformable skirt can be positively snapped into place over the neck portion of the drug vial or bottle to maintain the adapter in secure connection with the bottle.

Another object of the invention is to provide an apparatus as described in the preceding paragraphs, which is easy-to-use and functions to prevent the accidental separation of the length of tubing carrying the medicinal fluid from the bottle.

By way of summary, the vial access transfer set of the present invention, which is adapted for use with a conventional bottle or vial containing a medicinal fluid comprises a vial access adapter for interconnection with the bottle, the adapter comprising a top wall and a cannula connected to the top wall. The vial access adapter also includes a resiliently deformable, slitted skirt that is connected to the top wall and extends therefrom for telescopically receiving the upper neck portion of the bottle. The adapter skirt uniquely includes a generally cylindrical body portion, an angularly outwardly extending marginal portion and an angularly inwardly extending intermediate portion disposed between the body portion and the outwardly extending intermediate portion for releasably gripping the vial.

A length of tubing for carrying the medicinal fluid is connected at one end to a connector portion provided on the vial access adapter and a spike connector is connected at the opposite end of the length of tubing. In one form of the invention of the spike connector comprises a hollow body portion and a spike receiving means for sealably interconnecting the intravenous spike with said hollow body portion.

### BRIEF DESCRIPTION OF THE DRAWINGS

FIG. 1 is a generally diagrammatic view of a prior art vial access set and method of use thereof.

FIG. 1A is a greatly enlarged, generally perspective view of the area identified in FIG. 1 as “1A”.

FIG. 2 is a generally perspective, exploded view of one form of the vial access set of the present invention shown interconnected with a conventional medicinal vessel.

FIG. 3 is a greatly enlarged, cross-sectional view of one form of the vial access adapter of the apparatus of the invention for interconnecting the infusion line with the medicinal vessel.

FIG. 4 is a generally perspective bottom view of the vial access adapter shown in FIG. 3.

FIG. 5 is a greatly enlarged, cross-sectional view of the area designated in FIG. 3 as 5-5.

### DESCRIPTION OF THE INVENTION

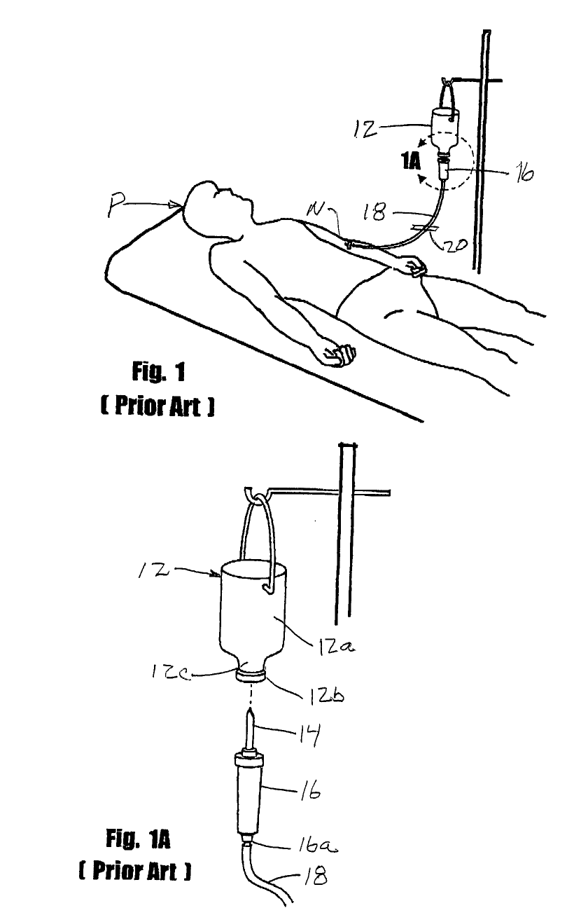

Referring to the drawings and particularly to FIGS. 1 and 1B, a common method of IV infusion of a medicinal fluid to a patient “P” is there illustrated. The prior art apparatus that is used comprises an IV container, such as an IV bottle 12, that contains the liquid to be infused into the patient. As indicated in FIG. 1A, the IV bottle or vial typically comprises a container portion 12a; an upper, generally cylindrically shaped portion 12b within which a pierceable, septum like member is sealably mounted and an intermediate neck portion 12c. In use, an IV spike 14 is used to penetrate the septum to establish a pathway through which the fluid can flow from the bottle to the patient. The spike 14 is generally connected to or formed integrally with the inlet port of a small, elongated, transparent hollow container 16 to form a fluid flow path between the medicament container or bottle 12 and the inlet port of the drip chamber via a fluid pathway provided within the spike.

To enable fluid flow to the patient “P”, an IV line, such is a length of flexible plastic tubing 18 and is connected to the outlet port 16a of the drip chamber. A flow control clamp such as a slide clamp 20 (FIG. 1) is typically engaged with the IV line to regulate fluid flow through the line. To complete the fluid flow path from the IV container to the patient, a needle “N” is connected to the IV line for insertion into the patient.

As illustrated in FIG. 1, during the infusion of the medicament or nutrient to the patient, bottle 12 is elevated above the patient to establish a positive pressure to force the fluid that is within the bottle 12 through the drip chamber 16 and into the patient “P”. Following the interconnection of the IV set to the patient the medical technician establishes a predetermined flow rate of medicament into the patient by adjusting the slide clamp 20 on the IV line 18.

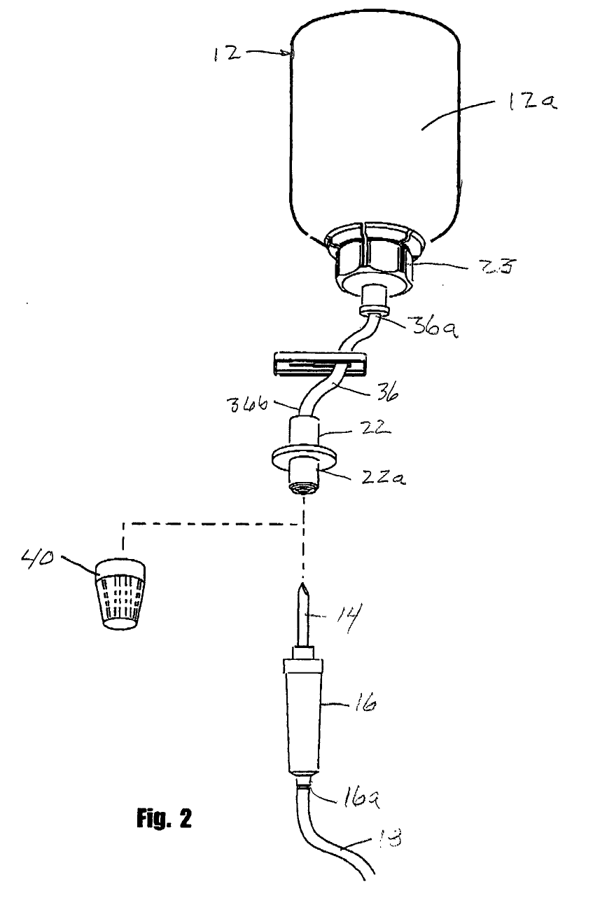

Turning now to FIG. 2 of the drawings, one form of the vial access transfer set of the present invention is there shown interconnected with a conventional fluid container such as a bottle 12 that contains the medicinal fluid to be delivered to the patient. Bottle 12 is of the construction previously described and includes a container portion 12a, an upper, generally cylindrically shaped portion and an intermediate neck portion. As previously mentioned, the vial access transfer set provides means for securely attaching an administration set to the bottle 12 and also provides a universal spike port 22, the construction of which will be presently described, to which any primary IV line can be readily attached.

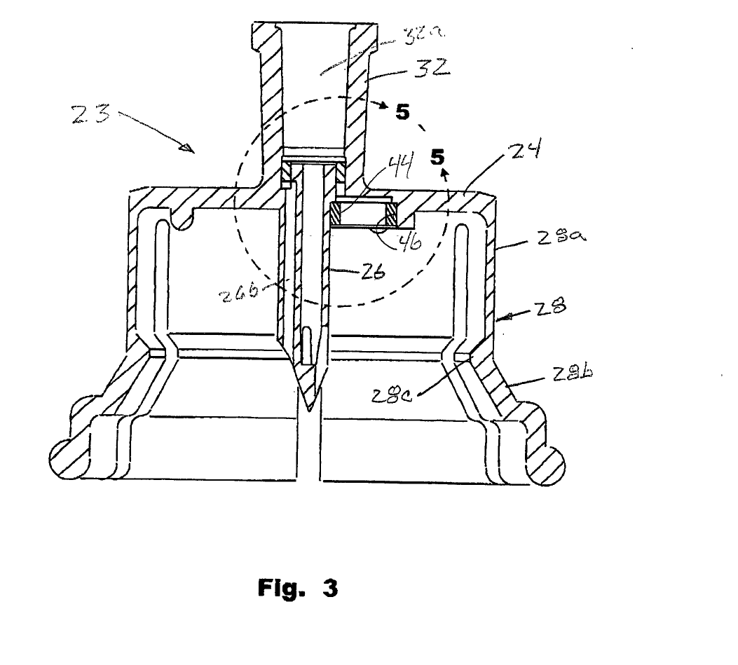

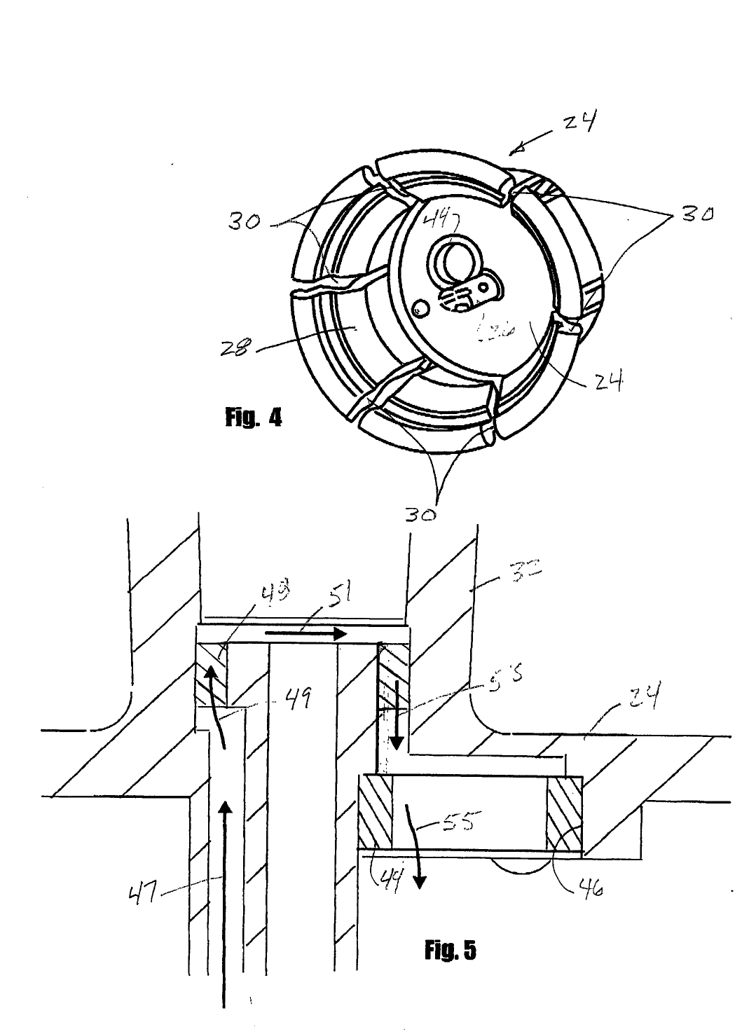

And important component of the vial access transfer set of the invention comprises a uniquely constructed, vented vial access adapter 23 for interconnection with the vial or bottle 12. As best seen by referring to FIG. 3 the vial access adapter, which is preferably formed of a moldable plastic, comprises a top wall 24, a hollow cannula 26 that is connected to top wall 22 and depends therefrom and a resiliently deformable skirt 28 that is connected to top wall 24. As indicated in FIG. 3, skirt 28 extends outwardly from top wall 24 and is adapted to telescopically receive the generally cylindrically shaped portion of bottle 12 in the manner illustrated in FIG. 2. The skirt 28 of the adapter is of a unique configuration and includes a generally cylindrical body portion 28a, an angularly outwardly extending marginal portion 28b and an angularly inwardly extending intermediate portion that is disposed between body portion 28a and intermediate portion 28b. This important intermediate portion comprises a circumferentially extending protuberance 28c that functions to releasably grip the neck portion of bottle 12 in a manner shown in FIG. 2. As best seen by referring to FIG. 4, skirt 28 of the vial access adapter of the present form of the invention is provided with a plurality of circumferentially spaced slits 30 that permit the skirt portion to flex sufficiently to enable it to be fairly effortlessly snapped over the neck portion of body 12.

Vented vial access adapter 23 further includes a connector portion 32 that is connected to top wall 24 and extends outwardly there from in the manner shown in FIGS. 2 and 3. Connector portion 32 functions to enable a length of tubing 36 to be connected proximate its first end 36a to the vial access adapter in the manner shown in FIG. 2. Connected proximate the second end 36b of tubing 36 is the previously identified universal spike port or spike connector 22. Spike connector 22, which is of a conventional construction, such as that commercially available from sources such as Borla of Italy, here comprises a hollow body portion 22a and a universal spike receiving means for sealably interconnecting an intravenous spike, such as spike 14 with the hollow body portion. Hollow body portion 22a includes a tapered bore that securely, sealably grips, not only spike 14, but also virtually any type of IV spike that is typically found on conventional IV primary lines. As previously mentioned, spike 14 is connected to or formed integrally with the inlet port of container 16 to form a fluid flow path between the medicament container or bottle 12 and the inlet port of the drip chamber via the fluid pathway within the spike. The assembly 60 as shown in FIG. 2, which is made up of spike 14 and container 16, is commercially available from various sources such as Borla in Italy, or Qosina in New York, N.Y.

A protective cap 40 is provided for covering hollow body portion 22a so as to maintain the hollow body portion in a substantially sterile condition until use.

Referring to FIGS. 3, 4 and 5, is to be noted that cannula 26, which is of a controlled, predetermined length, has a first lumen 26a that defines a fluid flow path between the interior of the bottle 12 and the flow passageway 32a of connector 32. Cannula 26 also has a second lumen 26b that defines a venting passageway for permitting the passage of air between the interior of the bottle 12 and atmosphere via appropriate air filtering means. This air filtering means here comprises a conventional microporous filter 44, which permits venting of gases through the filter but prevents the passage therethrough of liquid and particles of selected sizes. As previously mentioned, vial access adapter 23 is constructed of plastic and, therefore, is readily disposable, so as to avoid contamination from the adapter itself after it has been used.

As indicated in the drawings, filter 44 is mounted within a chamber 46 formed in top wall 24 of the adapter. With this construction, air can flow from the interior of the bottle 12 through lumen 26b in the direction of the arrow 47 and then through a conventional liquid filter 48 in the direction of arrow 49. Liquid filter 48, which is readily commercially available, permits the flow of air there through, but prevents the flow of liquid there through. After flowing through filter 48, the air then flows in the direction of arrows 51, 53 and 55 to atmosphere via air filter 44.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following documents.

We claim:

1. A vial access transfer set for use with a vial containing a medicinal fluid, said vial having a container portion; an upper, generally cylindrically shaped portion; and an intermediate neck portion, said vial access transfer set functioning to deliver the medicinal fluid from the vial to a patient via an intravenous spike connected to a delivery line, said vial access transfer set comprising:
(a) a vial access adapter for interconnection with the vial, said adapter comprising gripping means for releasably gripping the vial;
(b) a length of tubing connected to said vial access adapter; and
(c) a spike connector connected to said length of tubing, said spike connector comprising:
(i) a hollow body portion; and
(ii) a spike receiving means for sealably interconnecting the intravenous spike with said hollow body portion.

2. The vial access transfer set as defined in claim 1 in which said adapter comprises:
(a) a top wall;
(b) a cannula connected to said top wall;
(c) a resiliently deformable skirt connected to said top wall and extending therefrom for telescopically receiving the upper portion of the vial, said skirt having:
(i) a generally cylindrical body portion;
(ii) an angularly outwardly extending marginal portion; and
(iii) an angularly inwardly extending intermediate portion disposed between said body portion and said outwardly extending intermediate portion for releasably gripping the vial.

3. The vial access transfer set as defined in claim 2 in which said intermediate portion of said resiliently deformable skirt of said vial access adapter comprises a circumferentially extending protuberance formed intermediate said body portion and said angularly outwardly extending marginal portion.

4. The vial access transfer set as defined in claim 2 in which said skirt portion of said vial access adapter is provided with a plurality of circumferentially spaced slits.

5. The vial access transfer set as defined in claim 2 in which said vial access adapter further includes a connector portion connected to said top wall and extending therefrom.

6. The vial access transfer set as defined in claim 2 in which said vial access adapter is molded in a single piece from a moldable plastic.

7. The vial access transfer set as defined in claim 2 in which said vial access adapter includes a venting means for providing an air passageway between the interior of the vial and atmosphere.

8. The vial access transfer set as defined in claim 7 in which said cannula of said vial access adapter includes first and second spaced apart lumens and in which said venting means comprises a microporous filter disposed in communication with a selected one of said first and second spaced apart lumens.

9. The vial access transfer set as defined in claim 8 in which said microporous filter permits the passage of air there through, but prevents the passage of liquids there through.

10. The vial access transfer set as defined in claim 8 in which said vial access adapter further includes a liquid filter.

11. A vial access transfer set for use with a vial containing a medicinal fluid, said vial having a container portion; an upper, generally cylindrically shaped portion; and an intermediate neck portion, said vial access transfer set functioning to deliver the medicinal fluid from the vial to a patient via an intravenous spike connected to a delivery line, said vial access transfer set comprising:
(a) a vented vial access adapter for interconnection with the vial, said adapter comprising:
(i) a top wall having a cavity formed therein;
(ii) a microporous filter mounted within said cavity;
(iii) a connector portion connected to said top wall and extending therefrom;
(iv) a cannula connected to said top wall, said cannula having a first lumen in communication with said microporous filter and a second lumen in communication with said connector portion; and
(v) a resiliently deformable skirt connected to said top wall and extending therefrom for telescopically receiving the upper portion of the vial, said skirt having:
a. a generally cylindrical body portion;
b. an angularly outwardly extending marginal portion; and
c. an angularly inwardly extending intermediate portion disposed between said body portion and said outwardly extending intermediate portion for releasably gripping the vial.
(b) a length of tubing connected to said connector portion of vial access adapter; and
(c) a spike connector connected to said length of tubing, said spike connector comprising:
(i) a hollow body portion; and
(ii) a spike receiving means for sealably interconnecting the intravenous spoke with said hollow body portion.

12. The vial access transfer set as defined in claim 11 in which said intermediate portion of said resiliently deformable skirt of said vial access adapter comprises a circumferentially extending protuberance formed intermediate said body portion and said angularly outwardly extending marginal portion.

13. The vial access transfer set as defined in claim 11 in which said skirt portion of said vial access adapter is provided with a plurality of circumferentially spaced slits.

14. The vial access transfer set as defined in claim 11 in which said microporous filter permits the passage of air therethrough, but prevents the passage of liquids there through.

15. The vial access transfer set as defined in claim 11 in which said vial access adapter further includes a liquid filter disposed within said connector portion.
